(19) United States
(12) Patent Application Publication
(10) Pub. No.: US 2003/0032631 A1
(43) Pub. Date: Feb. 13, 2003

(54) Method of treatment of cancer by controlling graft-versus-leukemia using topical active corticosteriods

(76) Inventors: George B. McDonald, Bellevue, WA (US); Nicholas Stergiopoulos, Miami, FL (US)

Correspondence Address: LYON & LYON LLP, 633 WEST FIFTH STREET, SUITE 4700, LOS ANGELES, CA, 90071, US

(21) Appl. No.: 09/928,890

(22) Filed: Aug. 13, 2001

(51) Int. Cl. A61K 31/56

(52) U.S. Cl. 514/178; 514/179; 514/180

(57) ABSTRACT

A method for the improved treatment of blood-borne cancers, such as lymphomas, leukemia, and myeloma. The method comprises the oral administration of an effective amount of a topically active corticosteroid to a patient who has undergone hematopoietic cell transplantation. Administration of the TAC controls a graft-versus-leukemia (GVL) reaction that is induced following a hematopoietic cell transplantation, so that a GVHD reaction does not develop, or is reduced in severity. The GVL reaction effects killing of cancerous tumor cells in the blood, mediated by the cells derived from the hematopoietic cell transplantation.

### FIELD OF THE INVENTION

This invention relates to methods useful for the treatment of cancer. More particularly, this invention relates to methods that may be used in controlling a graft-versus-leukemia (GVL) reaction that accompanies an allogeneic blood or bone marrow transplant, so that graft-vs-host disease (GVHD) does not develop or is reduced in severity.

### BACKGROUND OF THE INVENTION

Leukemia, lymphoma and myeloma are cancers that originate in the bone marrow (in the case of leukemia and myeloma) or in lymphatic tissues (in the case of lymphoma). Leukemia, lymphoma and myeloma are considered to be related cancers, because they involve the uncontrolled growth of cells having similar functions and origins. The diseases result from an acquired (i.e., not inherited) genetic injury to the DNA of a single cell, which becomes abnormal (malignant) and multiplies continuously. The accumulation of malignant cells interferes with the body's production of healthy blood cells and makes the body unable to protect itself against infections.

Treatment of leukemia, lymphoma and myeloma usually involves one or more forms of chemotherapy and/or radiation therapy. These treatments destroy the malignant cells, but also destroy the body's healthy blood cells as well. Allogeneic bone marrow transplantation (BMT) is an effective therapy useful in the treatment of many hematologic malignancies. In allogeneic BMT, bone marrow (or, in some cases, peripheral blood) from an unrelated or a related (but not identical twin) donor is used to replace the healthy blood cells in the cancer patient. The bone marrow (or peripheral blood) contains stem cells, which are the precursors to all the different cell types (e.g., red cells, phagocytes, platelets and lymphocytes) found in blood. Allogeneic BMT has both a restorative effect and a curative effect. The restorative effect arises from the ability of the stem cells to repopulate the cellular components of blood. The curative properties of allogeneic BMT derive largely from a graft-versus-leukemia (GVL) effect. The hematopoietic cells from the donor (specifically, the T lymphocytes) attack the cancerous cells, enhancing the suppressive effects of the other forms of treatment. Essentially, the GVL effect comprises an attack on the residual tumor cells by the blood cells derived from the BMT, making it less likely that the malignancy will return after transplant. Controlling the GVL effect prevents escalation of the GVL effect into GVHD.

Blood is an increasingly frequent source of stem cells for transplantation. Under normal conditions, stem cells circulate in the blood in very small numbers. Drugs are available that increase the numbers of stem cells in the blood by drawing them out of the marrow. Sufficient quantities of these stem cells for transplantation are recovered by circulating large volumes of blood through a hemapheresis machine and skimming off a population of cells that contains stem cells. BMT, while originally referring specifically to bone marrow transplantation, more recently has become a generic term for blood or marrow transplantation, to encompass the use of blood transplantation as well as bone marrow transplantation. In many cases, the more specific term “stem cell transplantation” (or SCT) is now used.

However, allogeneic BMT is often toxic to the patient. The toxicity arises from the difficulty in dissociating the GVL effect from graft-versus-host disease (GVHD), an often-lethal complication of allogeneic BMT. Graft-versus-host disease (GVHD) is a complication of allogeneic hematopoietic cell transplantation in which tissues of the host, most frequently the skin, liver and intestine, are damaged by lymphocytes from the donor graft. When a patient has GVHD, treatment is successful only 50-75% of the time; the remainder of the patients generally die. The risk and severity of this immune-mediated condition are directly related to the degree of mismatch between a host and the donor of hematopoietic cells. For example, GVHD develops in up to 30% of recipients of human leukocyte antigen (HLA)-matched sibling marrow, in up to 60% of recipients of HLA-matched unrelated donor marrow, and in a higher percentage of recipient of HLA-mismatched marrow. Patients with mild intestinal GVHD present with anorexia, nausea, vomiting, abdominal pain and diarrhea, whereas patients with severe GVHD are disabled by these symptoms. If untreated, symptoms of intestinal GVHD persist and often progress; spontaneous remissions are unusual. In its most severe form, GVHD leads to necrosis and exfoliation of most of the epithelial cells of the intestinal mucosa, a frequently fatal condition. The separation of GVL from GVHD is therefore an extremely important therapeutic goal of BMT.

In treatment of GVHD, the objectives are to suppress a wide variety of biological events that result in tissue destruction, for example, the generation of inflammatory cytokines, the recruitment of additional inflammatory cells to the site of injury, the destruction of the barrier function of the intestinal mucosa (the lining), the passage of bacteria and toxins through the damaged intestinal mucosa, the up-regulation of biologic responses to bacteria and endotoxin, and the widespread organ responses to these events (such as leaky blood vessels, increased cardiac output, decreased systemic vascular resistance, diffuse lung injury, and renal insufficiency). Standard initial treatment for GVHD includes systemic immunosuppressive agents, usually high-dose prednisone at about 2 mg per kilogram body weight per day added to prophylactic medications such as methotrexate, cyclosporine and tacrolimus. Prednisone achieves a complete and sustained remission of gastrointestinal symptoms in 50-70% of patients with GVHD. Patients who fail to respond to the standard therapy may receive additional immunosuppressive regimes, such as higher-dose prednisone, anti-lymphocyte globulin, and investigational stage anti-T-cell monoclonal antibodies or immunotoxins. Unfortunately, the risks of prolonged immunosuppressive therapy are significant, especially among patients with immature bone marrow grafts. These risks include local and disseminated infection, the development of lymphoproliferative disease, and systemic glucocorticoid side effects such as hypothalamic-pituitary-adrenal axis suppression, myopathy, neuropsychiatric disease, and bone demineralization.

A related condition to GVHD is host-versus-graft disease (HVGD), also referred to as organ allograft rejection. HVGD disease may occur, for example, when a donor intestine is transplanted into a patient with a diseased intestine. In this case, cells from the patient's immune system (the host) may attack the foreign intestinal tissue (the graft). While intestinal transplantation is not routine at the present time, such techniques will likely become more common. Thus, prophylactic medications also are needed to prevent HVGD for many of the reasons noted above with regard to GVHD.

U.S. Pat. No. 6,096,731 (McDonald) describes a method for the treatment of GVHD or HVGD that comprises administration of a prophylactically effective amount of a topically active corticosteroid to a patient following intestinal or liver transplantation. The TAC is administered for a period of time effective to prior to presentation of symptoms associated with either GVHD or HVGD. However, no information was given relating to methods of treatment of cancer by controlling a GVL reaction.

The results of a phase I trial of a topically active corticosteroid, beclomethasone dipropionate (BDP), for the treatment of patients with intestinal GVHD have been reported (Baehr et al., Transplantation 60:1231-1238, 1995). In this trial, BDP capsules were given orally, 8 mg daily, half as enteric-coated capsules designed to dissolve in the alkaline pH of the upper small intestine, and half of the capsules that dissolve in the stomach. Significant improvement was found in the appetite, oral intake, nausea, and diarrhea over the course of therapy with oral BDP alone and with oral BDP in combination with prednisone. However, the time to improvement in patients receiving BDP as monotherapy was 7-10 days, which is longer than the response usually seen with prednisone therapy.

A drawback to the above regimen is that treatment is initiated with BDP only after presentation of symptoms of intestinal GVHD, with typical patient enrollment at a mean of 58 days post-transplant (i.e., ranging from day 21-231 after transplant). The difficulty with treatment after presentation of intestinal GVHD symptoms is that significant inflammation and/or damage to the intestine has already occurred prior to the initiation of therapy. Severe damage to the lining of the intestine is often fatal, as malnutrition, protein loss, and blood stream infections preclude regeneration of lining cells.

While significant advances have been made with regard to the treatment of GVHD following bone marrow transplantation, there is still a need in the art for improved methods for treating cancers by controlling the GVL effect and preventing the damage associated with the onset of GVHD. Such preventative methods should begin immediately following hematopoietic cell transplantation, and reduce tissue damage associated with the subsequent onset of GVHD. The present invention fulfills these needs and provides further related advantages.

### SUMMARY OF THE INVENTION

The present invention discloses a method for the improved treatment of blood-borne cancers, such as lymphomas, leukemia, and myeloma. The method comprises the oral administration of an effective amount of a topically active corticosteroid (hereinafter “TAC”) to a patient who has undergone hematopoietic cell transplantation. Administration of the TAC controls a graft-versus-leukemia (GVL) reaction that is induced following a hematopoietic cell transplantation, so that a GVHD reaction does not develop, or is reduced in severity. The GVL reaction effects killing of cancerous tumor cells in the blood, mediated by the cells derived from the hematopoietic cell transplantation. Such administration preferably begins at day 1 following the hematopoietic cell transplantation and preferably continues up to day 80 following the hematopoietic cell transplantation.

One aspect of the present invention comprises a method of treating an animal with cancer who has received an allogeneic hematopoietic cell transplant, comprising administering to the animal an amount of an oral topically active corticosteroid effective to prevent or reduce symptoms of GVHD while maintaining a GVL reaction effective to eliminate or reduce the number of cancer cells in the blood of the animal.

In another aspect of the present invention, there is provided a method of treating an animal who has received an organ allograft transplant, comprising administering to the animal an amount of an oral topically active corticosteroid effective to prevent or reduce symptoms of HVGD.

The above and other objects, features and advantages of the present invention will become apparent from the following description.

### DETAILED DESCRIPTION OF THE INVENTION

The present invention is directed to a method for the treatment of cancer by controlling a graft-versus-leukemia (GVL) reaction following hematopoietic cell transplantation. The method comprises the oral administration of an effective amount of a topically active corticosteroid (TAC) to a patient who has undergone, or immediately prior to undergoing, hematopoietic cell transplantation, so that a GVHD reaction does not develop, or is reduced in severity. As used herein, “hematopoietic cell transplantation” refers to bone marrow transplantation, peripheral blood stem cell transplantation, umbilical vein blood transplantation, or any other source of pleuripotent hematopoietic stem cells. The term “effective amount” refers to an amount of the TAC that reduces or eliminates one or more undesirable symptoms associated with graft-versus-host disease (GVHD) that arises as a consequence of the hematopoietic cell transplantation. The methods of the present invention are also useful in the treatment of host-versus-graft disease (HVGD) or allograft rejection which commonly follows organ transplantation. As used herein, the term “treatment” means administration of a therapy effective to reduce or control the GVL reaction of the symptoms between about day 1 and about day 80 following hematopoietic cell transplantation or HVGD following organ allograft transplantation. The term “patient” refers to any animal that may develop GVHD or HVGD, and will most often refer to a human.

Patients who may benefit from the methods of the present invention include those who have undergone or will undergo hematopoietic cell or organ allograft transplantation; those who are or will be allogenic hematopoietic cell recipients who have typically received marrow-ablative chemotherapy and/or total body irradiation followed by donor hematopoietic cell infusion; or patients who have undergone or will undergo intestinal or liver organ transplantation. Such procedures are well known to those skilled in this field, and the steps employed in these procedures do not form an element of the present invention.

An important aspect of the present invention is that the TAC is orally administered such that it is topically administered to the intestinal and/or liver tissue. Thus, oral administration, as that term is used herein, is intended to exclude any form of systemic administration, such as by intravenous injection. Oral administration ensures that the TAC has little (if any) systemic availability, but high topical activity on intestinal and/or liver tissue. Such limited distribution results in fewer side effects, which is a significant advantage of this invention.

By appropriate formulation of the TAC (such as enterically coated capsules), it can be delivered to all of the mucosal surface of the intestine and/or the liver in high doses. Thus, the TAC can achieve high concentrations in the intestinal mucosa where the initiating alloimmune recognition event is taking place. It is believed that blunting the initiating event prevents the large cascade of biologic events that make up the syndromes of GVHD and HVGD.

The method of the present invention employs oral administration of an effective amount of a topically active corticosteroid (TAC) to a patient who has undergone or will undergo hematopoietic cell or organ allograft transplantation. Representative TACs include, but are not limited to, beclomethasone 17,21-dipropionate, alclometasone dipropionate, busedonide, 22S busedonide, 22R busedonide, beclomethasone-17-monopropionate, clobetasol propionate, diflorasone diacetate, flunisolide, flurandrenolide, fluticasone propionate, halobetasol propionate, halcinocide, mometasone furoate, and triamcinalone acetonide. Such TACs are well known to those skilled in the field of, for example, intestinal disorders, and are commercially available from any number of sources. Suitable TACs useful in the practice of this invention are any that have the following characteristics: rapid first-pass metabolism in the intestine and liver, low systemic bioavailability, high topical activity, and rapid excretion (see, e.g., Thiesen et al., Alimentary Pharmacology & Therapeutics 10:487-496, 1996) (incorporated herein by reference).

In a preferred embodiment of this invention, the TAC is beclomethasone dipropionate(BDP). BDP(9-chloro-11β,17,21-trihydroxy-16β-methylpregna-1,4-diene-3,20-dione 17,21 dipropionate) has a chemical formula of C₂₈H₃₇ClO₇, and is available from a number of commercial sources, such as Schering-Plough Corporation (Kenilworth, N.J.) or Pharmabios in Italy in bulk crystalline form. BDP has the following structure:

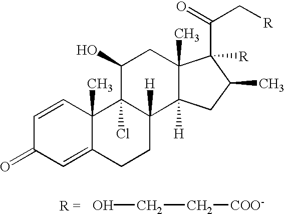

The TAC may be formulated for oral administration by techniques well known in the formulation field, including formulation as a capsule, pill, coated microsphere with specific dissolution qualities (i.e., a quick or slow-dissolving format), or emulsion. In the practice of this invention, at least two separate dosage forms of a TAC are administered to a patient in need thereof. The use of two different dosage forms allows the patient to receive TAC throughout the entire gastrointestinal tract, from the stomach to the rectum. It is preferable to limit the number of separate dosage forms to the smallest number possible; thus, two separate dosage forms is the preferred embodiment. The effective amount of TAC in each dosage form may vary from patient to patient, and may be readily determined by one skilled in the art by well-known dose-response studies. Such effective amounts will generally range between about 0.1 mg/day to about 8 mg/day, and more typically range from about 2 mg/day to about 4 mg/day. Accordingly, suitable capsules or pills generally contain from 1 mg to 2 mg TAC, and typically about 1 mg TAC, plus optional fillers, such as lactose, and may be coated with a variety of materials, such as cellulose acetate phthalate. By appropriate coating, such capsules, microspheres or pills may be made to dissolve within various location of the intestinal tract. For example, enteric-coated capsules prepared with a coating of cellulose acetate phthalate are known to dissolve in the alkaline environment of the small bowel, thus delivering its content to the small bowl and colon. Emulsions containing a TAC may also be employed for oral delivery, including optional emulsifying agents.

In addition to the TAC, acceptable carriers and/or diluents may be employed and are familiar to those skilled in the art. Formulations in the form of pills, capsules, microspheres, granules or tablets may contain, in addition to one or more TACs, diluents, dispersing and surface active agents, binders and lubricants. One skilled in the art may further formulate the TAC in an appropriate manner, and in accordance with accepted practices, such as those disclosed in Remington's Pharmaceutical Sciences, Gennaro, Ed., Mack Publishing Co., Easton, Pa., 1990 (incorporated herein by reference).

As optional components, other active agents may be administered in combination with the TAC, including (but not limited to) prednisone, prednisolone, cyclosporine, methotrexate, tacrolimus and biological agents that affect T-lymphocytes, such as anti-lymphocyte globulin, anti-T-cell monoclonal antibodies or anti-T-cell immunotoxins. Prednisone or prednisolone are preferably administered at a concentration of at least about 1 mg/kg body weight/day.

In the context of GVHD, long term therapeutic administration of a TAC preferably begins after the first day after infusion of hematopoietic cells, and continues for 80 days after infusion of hematopoietic cells.

Various patents and publications are cited herein, and their disclosures are hereby incorporated by reference in their entireties. The present invention is not intended to be limited in scope by the specific embodiments described herein. Although the present invention has been described in detail for the purpose of illustration, various modifications of the invention as disclosed, in addition to those described herein, will become apparent to those of skill in the art from the foregoing description. Such modifications are intended to be encompassed within the scope of the present claims.

We claim:

1. A method of treating an animal with cancer who has received an allogeneic hematopoietic cell transplant, comprising administering to said animal an amount of an oral topically active corticosteroid effective to prevent or reduce symptoms of GVHD while maintaining a GVL reaction effective to eliminate or reduce the number of cancer cells in the blood of said animal.

2. The method of claim 1, wherein said animal is a human.

3. The method of claim 1, wherein said topically active corticosteroid is administered in at least two different dosage forms.

4. The method of claim 1 wherein the topically active corticosteroid is administered orally at a dosage of between about 0.1 mg per day to about 8 mg per day.

5. The method of claim 1 wherein the topically active corticosteroid is administered orally at a dosage of between about 2 mg per day to about 4 mg per day.

6. The method of claim 1 wherein the topically active corticosteroid is administered orally from day 1 to about day 80 following hematopoietic cell transplantation.

7. The method of claim 1 wherein the topically active corticosteroid is administered in combination with prednisone or prednisolone at a concentration of at least 1 mg/kg body weight/day.

8. The method of claim 1 wherein the topically active corticosteroid is formulated for oral administration in the form of a pill, tablet, capsule or microsphere.

9. The method of claim 8 wherein the topically active corticosteroid is formulated such that the pill, microsphere, or capsule dissolves in the stomach, small intestine or colon.

10. The method of claim 1 wherein the topically active corticosteroid is formulated for oral administration in the form of an emulsion.

11. The method of claim 1 wherein said topically active corticosteroid is administered following infusion of the hematopoietic cells.

12. The method of claim 1 wherein administration of the topically active corticosteroid ceases after 80 days following infusion of the hematopoietic cells.

13. The method of claim 1 wherein the patient has received an allogeneic bone marrow transplant.

14. The method of claim 1 wherein the patient has received an allogeneic blood transplant.

15. The method of claim 1 wherein the topically active corticosteroid is administered in combination with at least one of cyclosporine, methotrexate, tacrolimus, anti-lymphocyte globulin, anti-T-cell monoclonal antibodies and anti-T-cell immunotoxins.

16. The method of claim 1 wherein the topically active corticosteroid is beclomethasone 17,21-dipropionate.

17. The method of claim 1 wherein the topically active corticosteroid is alclometasone dipropionate, busedonide, 22S busedonide, 22R busedonide, beclomethasone-17-monopropionate, clobetasol propionate, diflorasone diacetate, flunisolide, flurandrenolide, fluticasone propionate, halobetasol propionate, halcinocide, mometasone furoate, or triamcinalone acetonide.

18. A method of treating an animal who has received an organ allograft transplant, comprising administering to said animal an amount of an oral topically active corticosteroid effective to prevent or reduce symptoms of HVGD.
